(19) United States
(12) Patent Application Publication
(10) Pub. No.: US 2004/0199138 A1
(43) Pub. Date: Oct. 7, 2004

(54) Storage of liquid compositions

(76) Inventors: William Edward McBay, Fife (GB); Anne Gillian Welch, Fife (GB); Ronald Vance McIntosh, Edinburgh (GB); Peter Reynolds Foster, Edinburgh (GB)

Correspondence Address: MYERS BIGEL SIBLEY & SAJOVEC, PO BOX 37428, RALEIGH, NC, 27627, US

(21) Appl. No.: 10/484,515

(22) PCT Filed: Jul. 19, 2002
(86) PCT No.: PCT/GB02/03337

(30) Foreign Application Priority Data
Jul. 21, 2001 (GB) 0117879.7

(51) Int. Cl. A61B 19/00; A01N 37/18

(52) U.S. Cl. 604/403; 428/34.6; 514/2

(57) ABSTRACT

Liquid pharmaceutical compositions stored in glass containers, (particularly of blood plasma proteins such as albumin) having a shelf-life in excess of 18 months; over which time the aluminium content is maintained below 200 micrograms per litre. This is achieved by silicone-coating the glass container. Also the particles/ml post-autoclave and post-pasteurisation are kept to low levels.

The present invention relates to the storage of the liquid pharmaceutical compositions, particularly injectable parenteral compositions, in glass containers in such manner that the aluminium content thereof does not rise above acceptable limits over the storage period and/or whereby the level of particulates contained therein is kept to a minimum.

Aluminium present in injectable solutions is a known clinical problem. In trace amounts, aluminium has been implicated in disorders such as senile dementia of the Alzheimer type, dialysis encephalopathy and kidney damage. For this reason, regulatory authorities have set maximum limits for the aluminium content of injectable pharmaceutical products. For example, the aluminium content of human albumin (generally derived from blood donations) may not exceed 200 micrograms per litre and must be maintained below this level throughout the shelf life of the product. This, however, poses a problem since most injectable solutions are stored in glass containers. Plastic containers are generally not used because of the possibility of leaching plasticisers and unreacted monomer therefrom.

It is, however, known that glass containers tend to leach out aluminium into solutions in contact therewith over extended periods of time, particularly in the case of alkaline solutions. Glasses are typically alumino silicates. Various attempts have been made to address the problem of aluminium leaching from glass containers over the storage shelf life of injectable solutions.

Kabivitrum patent EP0484464 (WO91/00290) addresses the problem by reducing the initial aluminium content to low levels by diafiltration prior to storage. It is known that different types of glass (identified as type I, II and III) have different properties as regards aluminium leaching. The use of type II glass, which has been treated with sulphur dioxide or ammonium sulphate, is proposed in Green Cross patent application EP559895. However, glass types I and II have been implicated in producing increased aluminium levels in human albumin solutions on extended storage at ambient temperatures (Ray Victor et al, Transfusion 1988, Vol.28, No.3, page 290). Another approach to the maintenance of reduced aluminium levels is set out in Grifols patent application EP0787498, wherein the presence of citrate in albumin solutions is said to increase the rate of leaching of aluminium from glass. The Inventors of this patent application therefore propose the maintenance of reduced citrate levels as a way of mitigating aluminium leaching.

Type II (soft) glass has a lower aluminium content than type I (hard) glass. However, despite using type II glass, the present applicants have continued to experience problems of aluminium accumulation in 20% human albumin solutions, with the 200 microgram per litre limit being exceeded within 18 months.

The problem of particulates in injectable solutions is also a known problem and has been implicated in phlebitis (Allcutt et al., BR. J. Search. Vol. 70 (1983) 111-113). A review of the problem is given in Borchert et al., Journal of Parenteral Science & Technology, Vol.40, No.5/September-October, 1986. In particular, it is found that storage of injectable solutions in glass containers can increase the particulate content thereof, particularly when subject to autoclaving or pasteurisation.

It is an object of the present invention to mitigate the problems of aluminium leaching and/or particulate accumulation in such liquid compositions.

Generally speaking, it has been surprisingly found that siliconisation of glass storage containers mitigates both the problems of aluminium leaching and particle accumulation. Siliconised glass containers are commercially available for other purposes and are generally used to provide a non-wetting surface such that exactly predetermined doses can be dispensed.

Thus, in one aspect the present invention provides a method of storing a liquid pharmaceutical composition susceptible to uptake of aluminium leached from glass containers; which comprises storing the liquid composition in a glass container, whose surface contacting the liquid composition is coated with a silicone coating; whereby uptake of aluminium is mitigated.

Another aspect of the invention provides a storage container which comprises a glass container containing a liquid pharmaceutical composition, the surface of the glass container contacting the liquid composition being coated with a silicone coating.

The liquid pharmaceutical composition is generally an injectable solution which is generally apyrogenic and may have been sterilised by autoclaving or pasteurisation. However, the liquid composition may in principle be any pharmaceutical composition including oral or parenteral compositions, where there is a requirement for reduced aluminium levels to be maintained on storage.

There is a direct relationship between aluminium levels and storage period. Increase in temperature promotes leaching. Generally speaking, the aluminium content should be maintained below 200 micrograms per litre, preferably 150 micrograms per litre, particularly less than 100 micrograms per litre and especially less than 50 micrograms per litre over a storage period (i.e. shelf life) of 12 months, 18 months, 24 months, 36 months, and even up to 48 months; at ambient temperature.

Generally, the pH of the liquid composition will be in the range 4 to 10. Alkaline solutions generally exacerbate the problem of aluminium leaching and are preferably avoided.

The liquid pharmaceutical composition is generally a solution. The present invention is particularly applicable to the storage of blood-derived products, including those derived from human blood donations and those which are produced by recombinant DNA technology. This includes various preparations of plasma proteins such as albumin, plasma protein fraction, transferrin, immunoglobulins, fibrinogen, thrombin, factor VIII, factor IX, factor VII, factor VIIa, von Willibrand factor, factor XIII, antithrombin III, alpha-1-antitrypsin, CI-inhibitor, mannose binding lectin and other protein compositions. However, the invention is in principle applicable to any other liquid pharmaceutical composition where there is a need to maintain low aluminium content.

Aluminium leaching may also be reduced, as is known, by maintaining a lo:w citrate concentration in the liquid composition, for example less than or equal to 0.2 mmol/L, especially less than or equal to 0.1 mmol/L and particularly less than or equal to 0.05 mmol/L.

In particular, at a citrate content of less than 0.25 mmol/L an aluminium content of less than 50 μmol/L can be maintained at ambient temperature for at least 30 months. At a citrate content of less than 0.1 mmol/L an aluminium content of less than 40 μmol/L can be maintained at ambient temperature for at least 24 months.

With the siliconisation treatment of the present invention, the type of glass used becomes less critical. Thus, although it is preferred to use soft type II glass, hard type I glass or type III may also be employed.

The siliconisation treatment applied to the glass according to the present invention can be carried out in known manner using any available silicone polymer or prepolymer. The silicone coating can be deposited from solution or suspension or may be created by in situ polymerisation. The coating can be baked at elevated temperature e.g. 300° to 400° to harden the coating and remove extractables. The application of silicone coatings is described, for example, in “Siliconisation of Parenteral Drug Packaging Components” J. Parenteral Sci. Technol. (1988) Vol.42, Suppl.4S, S3-S13; and in Mundry et al., PDA Journal of Pharmaceutical Science & Technology, Vol.54, No.5, September/October 2000. The silicone may be any of those known to be suitable and pharmaceutically acceptable and in particular is a polydimethylsiloxane (linear or cyclic polymer) of the following general formula:

(CH₃)₃SiO[SiO(CH₃)₂]ₓSi(CH₃)₃

where x for linear polymers can be two to several thousand (e.g. 2 to 5000, particularly 10 to 1000, especially 100 to 500); while x for cyclic polymers is usually 3 to 14 with 4 and 5 being most common. The heat-cured silicones generally have a number average molecular weight or weight average molecular weight in excess of 10,000, possibly in excess of 20,000 and generally in the range 15,000 to 25,000.

It is found that the siliconisation treatment of the glass container also reduces the number of particles shed from the container surface—leading to reduced particulates in the liquid composition. Previous British Pharmacoepia Standards set limits of 1,000 particles/mL (for particles greater than or equal to 2 microns) and 100 particles/mL (for particles greater than or equal to 5 microns). These limits are achievable even post-autoclaving or post-pasteurisation employing the siliconised containers of the present invention. Typically, post-autoclaving the present invention is able to achieve less than 1,000, (generally less than 700) particles per ml for greater than or equal to 2 micron particle size; and less than 75 (generally less than 50) particles per ml for greater than or equal to 5 micron particle size. Post-pasteurisation values achievable are less than 500 particles per ml for greater than or equal to 2 micron particle size and less than 30 particles per ml for greater than or equal to 5 micron particle size.

Thus, the present invention allows aluminium content of liquid compositions to be maintained over an extended shelf life, without the need to resort to any special changes in processing procedures for blood products nor the need to include formulation additives.

Whilst the present invention has been described in relation to the mitigation of aluminium leaching from glass containers, it is equally applicable to the leaching of other metal ions, particularly multivalent metal ions, such as chromium, iron, manganese and nickel.

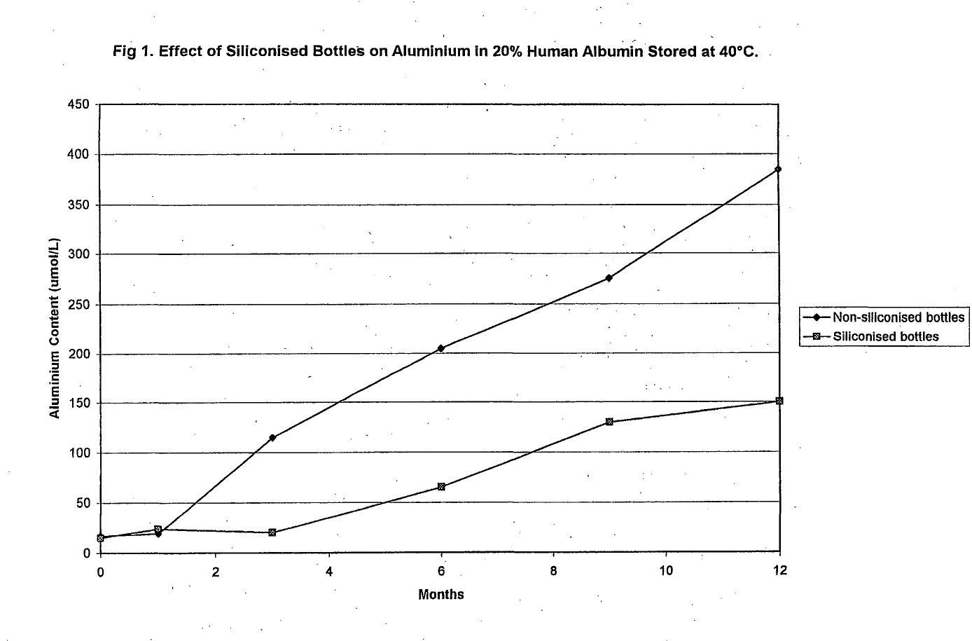

Embodiments of the present invention will now be described by way of example only in the following examples with reference to attached FIGS. 1 to 4.

The following Examples relate to the use of siliconised bottles to (a) reduce the contamination of albumin with aluminium during storage in glass bottles and (b) reduce the level of particulate matter in parenteral solutions in glass containers.

### EXAMPLE 1

Human plasma was processed by cold-ethanol, (Cohn) fractionation to obtain fraction V precipitate in which ≧95% of the total protein was albumin. Fraction V precipitate was resuspended in purified water to a total protein concentration of 80 g/L and clarified by depth filtration. The clarified solution was concentrated by ultrafiltration to a protein concentration of 120 g/L, diafiltered against 5 volumes of sodium chloride solution (132 mmol/L) to reduce the ethanol concentration to ≧8 mg/g protein and then concentrated by ultrafiltration to a protein concentration of about 220 g/L.

The resultant solution was stabilised by the addition of sodium octanoate and adjusted to a protein concentration of 200 g/L prior to membrane filtration to 0.2 μm. Aliquots of the filtered solution were dispensed aseptically into two types of glass container, either:

(a) 100 mL white type II DIN infusion bottles (supplied by International Bottle Company Ltd), or

(b) 100 mL white type II DIN infusion bottles with silicone treatment (supplied by International Bottle Company Ltd.). These bottles had been treated with a silicone emulsion, Pharasil E1049 (a 35% oil-in-water emulsion of polydimethylsiloxan) which was sprayed into each bottle.. The bottles had then been heated at 400° C. in order to fix the silicone to the surface of the glass container.

Following aseptic dispensing of the albumin solution, all bottles were sealed hermetically, subjected to a process of pasteurisation at 60° C. for 10 hours and then incubated at 31° C. for 14 days.

The citrate content of the Human Albumin product was determined by the enzymatic method of Möllering and Gruber (Analytical Biochemistry 1966; 17: 369-376) using a kit supplied by Boehringer Mannheim. Aluminium was determined by inductively-coupled plasma emission spectroscopy (ICP) using the aluminium line at 167 nm.

The aluminium content of the Human Albumin in each type of bottle, ie. container (a) non-siliconised or container (b) siliconised, was monitored during storage at 40° C. as an accelerated test of stability and at 25° C. as a test of room temperature stability.

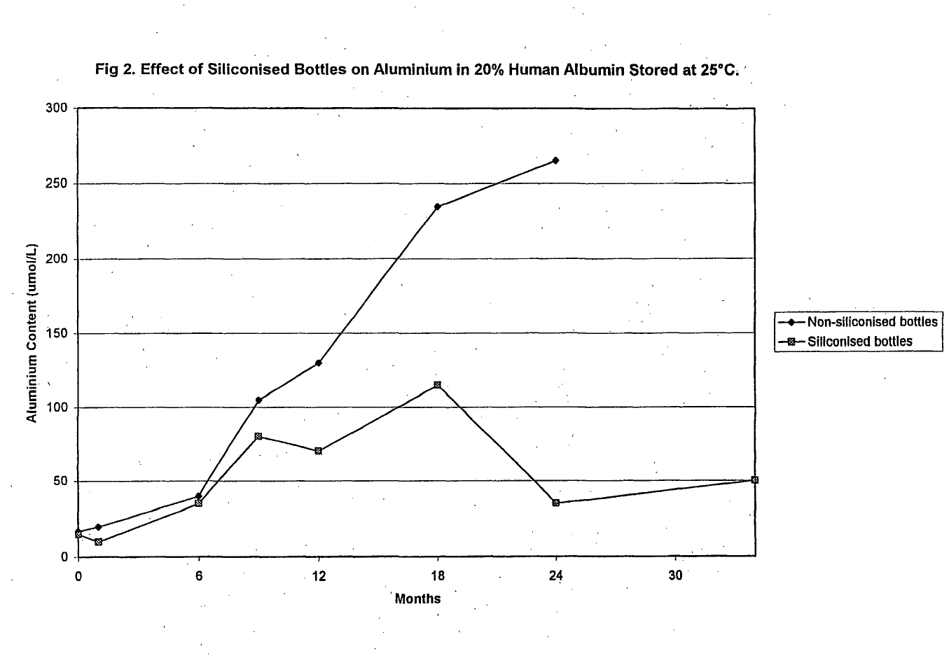

The concentration of citrate in the final solution of Human Albumin was 0.25 mmol/L. The concentrations of aluminium at different time points during storage are shown graphically in FIG. 1 for storage at 40° C. and in FIG. 2 for storage at 25° C. (the 34 month value for the siliconised bottle is unavailable).

It is evident from these results that the aluminium content of the Human Albumin solution was substantially lower in the siliconised bottles than in the non-siliconised bottles, both at room temperature storage (25° C.) and under the accelerated test of storage (40° C.).

### EXAMPLE 2

Human albumin was prepared as in Example 1, except that the diafiltration volume was increased from 5 volumes of sodium chloride solution (132 mmol/L) to 7 volumes of sodium chloride solution (132 mmol/L) in order to obtain a lower concentration of citrate in the final product.

The solution of Human Albumin (reduced citrate) was dispensed into non-siliconised and into siliconised bottles as described in Example 1, subjected to pasteurisation at 60° C. for 10 hours and incubation at 31° C. for 14 days prior to testing for stability at 40° C. and at 25° C.

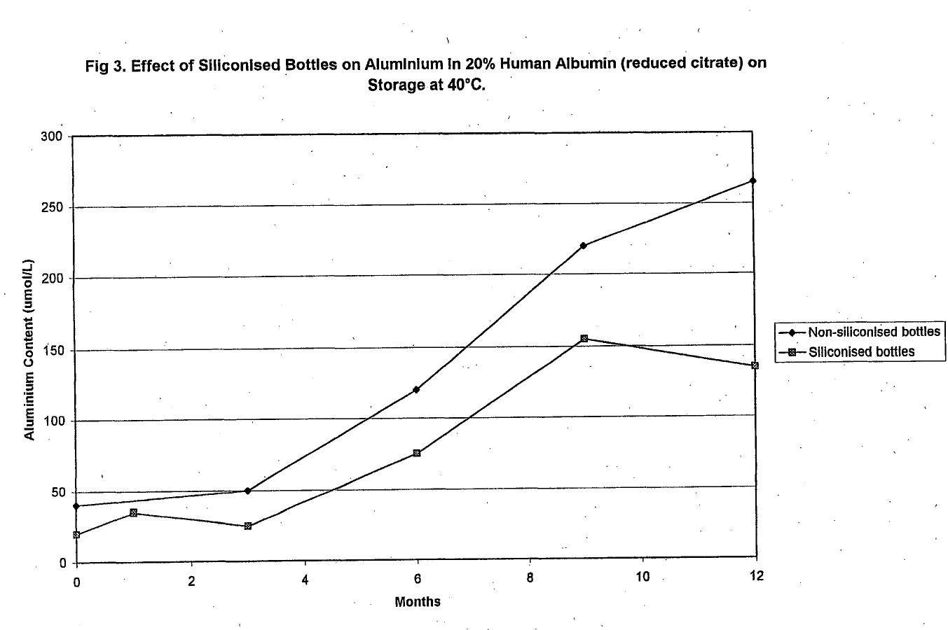

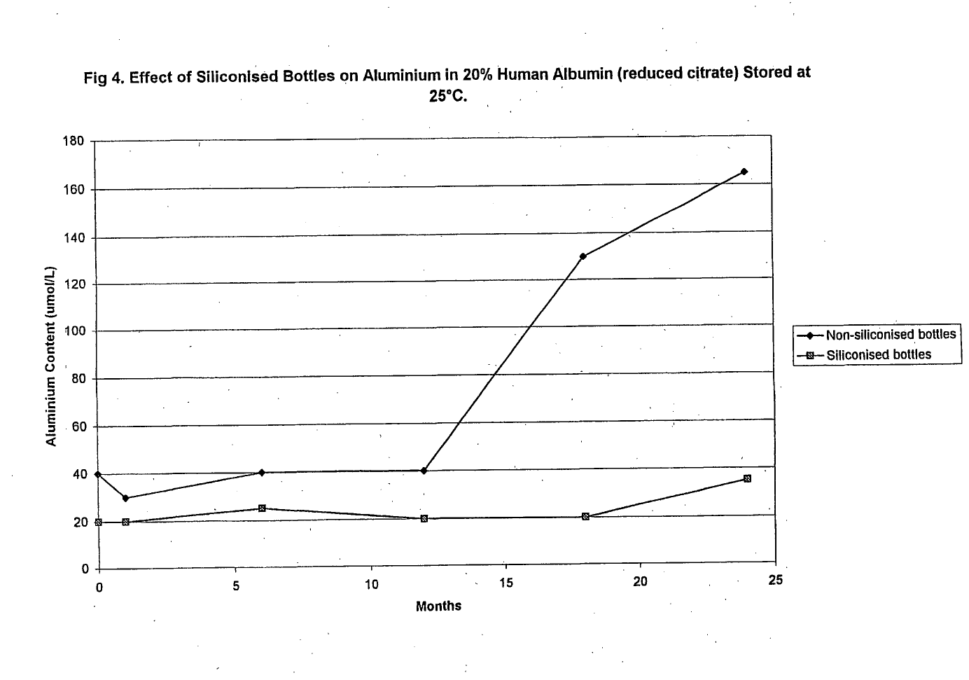

The citrate concentration of Human Albumin (reduced citrate) prepared in this manner was 0.1 mmol/L. The aluminium concentrations at different time points during storage are shown graphically in FIG. 3 for storage at 40° C. and in FIG. 4 for storage at 25° C.

In this experiment, despite the lower concentration of citrate in the protein solution, the Human Albumin (reduced citrate) stored in siliconised bottles exhibited a lower aluminium content than the same batch of product stored in non-siliconised bottles, both under accelerated storage (40° C.) and at room temperature storage (25° C.).

### EXAMPLE 3

The presence of particulate matter was determined in samples of Water For Injection. Samples of Water For Injection were collected into a particle free cuvette (Accuvette, Coulter Ltd) prior to the product being dispensed into bottles.

The Water For Injection was dispensed into either:

(a) sterile 500 mL bottles of type II glass, or

(b) sterile 500 mL bottles of type II glass which had been subjected to a silicone treatment. The silicone treatment involved immersion of the bottles in a siliconising solution, rinsing to remove excess silicone and then fixing of the silicone to the glass surface by baking the bottles at 110° C. for 16 hours.

After dispensing the Water For Injection; the bottles were hermetically sealed and the water terminally sterilised by autoclaving for 16 minutes at 121° C. under a pressure of 1.15 bar.

Samples from the different bottles were taken into particle free cuvettes (Accuvette, Coulter Ltd) and particle counts/mL were determined semi-automatically for particles ≧2 μm and for particles ≧5 μm according to the electrical zone-sensing method (Coulter Counter).

The results are summarised in Table 1. The data demonstrate an increase in the number of particles after dispensing into standard (non-siliconised) glass bottles and a further increase in the number of particles following sterilisation by autoclaving at 121° C. By contrast, with siliconised bottles, there was little increase in particulate matter after dispensing into the glass bottle and a smaller increase in the number of particles after heat sterilisation at 121° C. The overall effect was a much lower degree of particulate contamination in Water For Injection contained in siliconised glass bottles compared with that contained in non-siliconised glass bottles.

TABLE 1
Particles present in Water For Injection using non-siliconise
and siliconised bottles

| | | | particles/ | Particles/ |
|---|---|---|---|---|
| container | stage | n | mL, ≧2 μm | mL, ≧5 μm |
| particle free cuvette | pre-dispensing | 5 | 143 ± 52 | 24 ± 11 |
| non-siliconised bottle | pre-autoclave | 12 | 490 ± 169 | 59 ± 27 |
| siliconised bottle | pre-autoclave | 3 | 183 ± 73 | 21 ± 13 |
| non-siliconised bottle | post-autoclave | 11 | 1266 ± 398 | 122 ± 57 |
| siliconised bottle | post-autoclave | 3 | 362 ± 169 | 45 ± 23 |

n = number of batches tested

### EXAMPLE 4

As Example 3, using a sterile solution of 9 g/L sodium chloride (Saline) instead of Water For Injection. Table 2 shows the number of particles determined in the different samples taken. As in Example 3, the use of siliconised bottles resulted in little increase in particulate matter after dispensing into the glass bottle and a much lower number of particles after heat sterilisation of the Saline solution at 121° C.

TABLE 2
Particles present in Saline using non-siliconised and
siliconised bottles.

| | | | particles/ | particles/ |
|---|---|---|---|---|
| Container | stage | n | mL, ≧2 μm | mL, ≧5 μm |
| particle free cuvette | pre-dispensing | 2 | 106 | 10 |
| non-siliconised bottles | pre-autoclave | 7 | 575 ± 426 | 52 ± 40 |
| siliconised bottles | pre-autoclave | 3 | 125 ± 25 | 11 ± 4 |
| non-siliconised bottles | post-autoclave | 12 | 1301 ± 332 | 111 ± 25 |
| siliconised bottles | post-autoclave | 3 | 606 ± 208 | 45 ± 6 |

n = number of batches tested

### EXAMPLE 5

As Example 4, except that the bottled Saline was subjected to a process of pasteurisation at 60° C. for 10 hours instead of heat sterilisation at 121° C. This was done to simulate the heat treatment that had been applied to solutions of human albumin in Example 1 and Example 2 above in order to examine the effect that this treatment might have on particulate contamination.

The results are summarised in Table 3. Although the impact of pasteurisation at 60° C. for 10 hours was less pronounced than autoclaving at 121° C., the degree of particulate contamination was lower when siliconised bottles were used compared with non-siliconised bottles.

TABLE 3
Particles present in pasteurised Saline using non-
siliconised and siliconised bottles.

| | | | particles/ | particles/ |
|---|---|---|---|---|
| container | stage | n | mL, ≧2 μm | mL, ≧5 μm |
| non-siliconised | pre-pasteurisation | 3 | 519 ± 74 | 53 ± 19 |
| bottles | | | | |
| siliconised bottles | pre-pasteurisation | 3 | 259 ± 202 | 17 ± 11 |
| non-siliconised | post-pasteurisation | 3 | 667 ± 292 | 49 ± 8 |
| bottles | | | | |
| siliconised bottles | post-pasteurisation | 3 | 393 ± 209 | 20 ± 11 |

n = number of batches tested

1. A method of storing a liquid pharmaceutical composition susceptible to uptake of aluminium leached from glass containers; which comprises storing the liquid composition over a storage period of 12 months at ambient temperature in a glass container, whose surface contacting the liquid composition is coated with a silicone coating;
whereby uptake of aluminium is mitigated such that the aluminium content is maintained below 200 micrograms per litre.

2. A method according to claim 1 wherein the liquid pharmaceutical composition is an injectable solution.

3. A method according to claim 1 wherein the liquid pharmaceutical composition comprises human albumin.

4. A method according to claim 1 wherein said aluminium content is maintained over a storage period of 18 months at ambient temperature.

5. A method according to claim 4 wherein the storage period is 24 months.

6. A method according to claim 4 wherein the storage period is 36 months.

7. A method according to claim 4 wherein the storage period is 48 months.

8. A method according to claim 1 wherein the pH of the liquid composition is in the range 4 to 10.

9. A method according to claim 2 wherein the liquid pharmaceutical composition comprises a blood plasma protein.

10. A method according to claim 1 wherein the liquid composition has a citrate concentration of equal to or less than 0.1 mmol/L.

11. A method according to claim 1 wherein the glass container is formed of a type II glass.

12. A method according to claim 1 wherein the silicone coating is produced from a polydimethylsiloxane which has been heated at 400° C.

13. A method according to claim 1 wherein the liquid composition has less than 700 particles of size greater than or equal to 2 microns per ml. post-autoclaving.

14. A method according to claim 1 wherein the liquid composition has less than 50 particles of size greater than or equal to 5 microns per ml. post-autoclaving.

15. A method according to claim 1 wherein the liquid composition has less than 500 particles of size greater than or equal to 2 microns per ml. post-pasteurisation.

16. A method according to claim 1 wherein the liquid composition has less than 30 particles of size greater than or equal to 5 microns per ml. post-pasteurisation.

17. A storage container which comprises a glass container containing a liquid composition comprising a blood plasma protein and having a shelf life of at least 18 months at ambient temperature, over which time the aluminium content thereof is maintained below 200 micrograms per litre; the surface of the glass container contacting the liquid composition being coated with a silicone coating.

18. A container according to claim 17 wherein the blood plasma protein is human albumin.
